(19) United States
(12) Patent Application Publication
(10) Pub. No.: US 2004/0248223 A1
(43) Pub. Date: Dec. 9, 2004

(54) Reduction of interfering peroxidase activity in peroxidase based detection methods

(75) Inventor: Ralph Magnotti, Biddeford, ME (US)

Correspondence Address: Patrick G. Gattari, McDonnell Boehnen Hulbert & Berghoff, 32nd Floor, 300 S. Wacker Drive, Chicago, IL, 60606, US

(73) Assignee: IDEXX Laboratories, Inc.

(21) Appl. No.: 10/455,140

(22) Filed: Jun. 5, 2003

(51) Int. Cl. G01N 33/53; G01N 33/537; G01N 33/543; C12N 9/08

(52) U.S. Cl. 435/7.92; 435/192

(57) ABSTRACT

Reagent, method and kit for reducing the interfering peroxidase activity in a method of detecting an analyte in a sample where the method uses a peroxidase as a component of a signal producing system. The reagent includes a peroxide, a chromogen and sodium azide. The method includes the use of the reagent in a detection method.

### BACKGROUND OF THE INVENTION

1. Field of the Invention

The invention is related to the reduction of interfering peroxidase activity in detection methods that use peroxidase as a component of a signal producing system. More specifically, invention is related to the use of an azide to reduce the interference of peroxidases present in a sample in methods for detecting analytes in the sample where the method uses peroxidase as a component of a signal producing system.

2. Description of Related Art

Peroxidases, most commonly horseradish peroxidase (HRP), are enzymes that are used in methods of detecting analytes in biological samples. Example of such methods include histochemical and immunoassay techniques. HRP is widely used for analysis, especially in concert with antibodies or antigens, due to its low cost, high sensitivity, versatility and convenience. Whole blood and saliva are examples of convenient biological samples and, for that reason, are increasingly being used for analysis, especially for point-of-care, rapid, or portable testing systems. Such samples, however, often contain peroxidases that interfere with the detection of the analyte using a peroxidase, such as HRP, as a member of a signal producing system.

Mammalian blood peroxidase activity is almost entirely due to the combined activities of eosinophil peroxidase (EPO), myeloperoxidase (MPO) and hemoglobin (Hgb). Eosinophils, neutrophils and erythrocytes can infiltrate tissue samples, producing interference from EPO, MPO, and Hgb, respectively. The blood of many vertebrates, including human, canine, horse, guinea pig, and mouse contain one or more of these oxidases which would be expected to produce an interference in detection methods using a peroxidase, such as horseradish peroxidase.

### SUMMARY OF THE INVENTION

In one aspect, the invention is directed to a reagent for use in a detection method having peroxidase as a component of a signal producing system. The reagent includes a peroxide or a peroxide forming compound, a chromogen and sodium azide.

In another aspect, the invention is directed to a signal producing system for use in an analyte detection method. The signal producing system includes a peroxidase, a chromogen and sodium azide.

In a further aspect, the invention is directed to a method of reducing interfering peroxidase activity in a method of detecting an analyte using a peroxidase as a component of a signal producing system. The method comprises using a reagent comprising a substrate for the peroxidase and sodium azide.

In yet another aspect, the invention is directed to a method of reducing interference of at least one animal peroxidase in an assay of a biological sample having horseradish peroxidase as a component of a signal producing system. The method includes adding to the sample a reagent comprising hydrogen peroxide, a chromogen and sodium azide.

Still further, the invention is directed to a method of detecting an analyte in a sample. The method includes conjugating HRP to a specific binding partner for the analyte, contacting the conjugate with the sample to form a complex including the conjugate bound to the analyte; separating the complex from unbound conjugate; contacting the complex with a reagent comprising a peroxide, a chromogen and sodium azide; and detecting a signal produced as a result of the activity of the HRP, thereby detecting the analyte in the sample.

In an additional aspect, the invention is directed to a method of detecting an analyte in a sample. The method includes conjugating HRP to an analog of the analyte, contacting the conjugate with the sample and a specific binding partner for the analyte to form a complex of the conjugate bound to the binding partner, separating the complex from unbound conjugate, contacting the complex with a reagent including a peroxide, a chromogen and sodium azide, and detecting the presence, absence or amount of a signal produced as a result of the activity of the HRP, thereby detecting the analyte in the sample.

In one more aspect, the invention is directed to a kit for detecting an analyte in a sample. The kit has reagent that includes a peroxide or a peroxide forming compound, a chromogen and sodium azide.

### DETAILED DESCRIPTION

The ability of peroxidase to catalyze the oxidation of a number of organic compounds in the presence of peroxide, resulting in formation of colored end-products, is utilized in several methods of determination. In one aspect, the invention provides a reagent for use in a detection method having horseradish peroxidase as a component of a signal producing system. The reagent includes a peroxide or a peroxide forming compound, a chromogen and sodium azide. A detection method is any method used to detect analytes, usually in biological samples such as whole blood, blood components, urine, saliva, tissue, etc. An analyte is a compound or composition to be measured, or the material of interest. The analyte may be a member of a specific binding pair (sbp) and may be a ligand, which is mono- or polyvalent, usually antigenic or haptenic, and is a single compound or plurality of compounds that share at least one common epitopic or determinant site. Detection methods also include the determination of analytes in non-biological samples such as environmental, agricultural or industrial samples. These examples are not intended to be limiting in any way and the scope of the detection method in which the reagent of the invention may be used is limited only to the extent that a peroxide is used as part of a signal producing system in the detection method. To the extent that samples to be analyzed contain interfering peroxidases, the reagent of the present invention provides means for reducing the interference of those peroxidases.

The present invention is advantageous in that unadulterated samples of tissue and body fluids such as blood, saliva, milk and urine may be used for the determination of analyte concentration, eliminating the need for costly and problematic (particularly for a rapid immunoassay device) sample processing steps. According to one aspect of the invention, hydrogen peroxide is either present in the reagent or is subsequently generated during the course of the detection method or assay. For example, the hydrogen peroxide may be used as an oxidizing agent, e.g., to oxidize a dye (chromophore) or other intermediate to generate a detectable species.

The signal producing system utilized as part of the detection method may have one or more components, at least one component being a peroxidase, such as HRP. The signal producing system generates a signal that relates to the presence, absence or amount of analyte in a sample. The signal producing system includes all of the reagents required to produce a measurable signal. For purposes of the invention, the signal-producing system includes at least a peroxidase and at least one substrate, and may include HRP or one or more peroxidases and a plurality of substrates, and may include a combination of enzymes, where the substrate of one enzyme is the product of the other enzyme; provided throughout that at least one enzyme of the signal producing system is a peroxidase. Other components of the signal producing system can include substrates, enhancers, activators, chemiluminescent compounds, cofactors, inhibitors, scavengers, metal ions, specific binding substances required for binding of signal generating substances, coenzymes, substances that react with enzyme products, other enzymes and catalysts, and the like. The signal producing system provides a signal detectable by external means, normally by measurement of electromagnetic radiation, desirably by visual examination. In one aspect of the invention, the signal producing system includes a chromophoric substrate and a peroxidase, where chromophoric substrates are enzymatically converted to dyes that absorb light in the ultraviolet or visible region.

As used herein “chromogen” is synonymous with chromophoric substrate and refers to a compound that becomes a colored compound upon reaction with a peroxidase. These compounds are widely known to those of skill in the art. The chromogen of the invention must be compatible with azide. Suitable non-limiting examples include TMB, o-phenylenediamines, 2,2′-azine-bis(3-ethylbenzothiazoline-6-sulfonic acid), Trinder reagents such as alkylanilines or phenols coupled to aminoantipyrine or 3-methyl-2-benzothiazolinone hydrazone, di- and tetra-aminobenzidines, aminoethylcarbazole, 4-chloronaphthol, and guaiacol.

Peroxidase is an enzyme catalyzing the oxidation of a number of chromophoric substrates in the presence of a peroxide. Peroxidases useful in the present invention include plant and fungal peroxidases, as these peroxidases are known to be selectively different than animal peroxidases. For example, animal peroxidases are substantially inhibited by azide whereas, as shown in the present invention, plant peroxidases retain activity in the presence of azide and are useful in the detection methods of the invention. As a plant peroxidase, HRP exists in the form of several isozymes, all containing heme as the prosthetic group. This enzyme has a molecular weight of approximately 40,000. Since HRP is similar to several other plant peroxidases, for example soybean peroxidase, and to fungal peroxidases, it is expected these other peroxidases will retain activity in the presence of sodium azide if used in a detection method and are therefore within the scope of the invention. Peroxidase enzymes may be either natural enzymes or enzymes produced by recombinant methods.

Peroxidase has been found well suited for the preparation of enzyme conjugated proteins, such as antibodies and antigens, due to its ability to yield chromogenic products, and to its relatively good stability characteristics. Peroxidase-labeled immuno-globulins have been used successfully as immuno-histological probes for the demonstration of tissue antigens, and in enzyme amplified immunoassay systems for the quantitative determination of soluble and insoluble antibodies and antigens. The use of the highly specific, sensitive and very stable horseradish peroxidase with a chromogenic donor has proven very useful for assay systems having or producing hydrogen peroxide.

Peroxides include hydrogen peroxide, alkylperoxide, polyolperoxide, urea peroxide and others that may be effectively used in detection method wherein the peroxide is oxidized by the peroxidase of the signal producing system. A peroxide forming compound is a compound capable of generating a peroxide—any substance other than molecular oxygen that is capable of producing a peroxide either directly or through the formation of one or more intermediates capable of producing a peroxide. Compounds that can be converted to, or can catalyze the formation of, a peroxide or exist as, or can be converted to, a catalyst for the formation of a peroxide can be detected as analytes in the method of this invention and are included in the above definition of “peroxide forming compound.”

For example, the present invention can utilize enzymes such as phosphatase, amylase, cholinesterase, creatinine kinase, and the like, and enzyme substrates such as glucose, cholesterol, creatinine, uric acid, and the like. These enzymes can be analytes themselves or simply a peroxide forming compound as part of the invention. The following discussion illustrates, by way of example and not limitation, the above, wherein a peroxide, i.e., hydrogen peroxide, is produced either directly or indirectly through the formation of one or more intermediate products in a system that ultimately produces hydrogen peroxide.

1) Amylase catalyzes the reaction of its substrate starch to form the intermediate product glucose, a substrate for glucose oxidase (1.1.3.4), which in the presence of molecular oxygen catalyzes the conversion of glucose to gluconic acid wherein hydrogen peroxide is also produced.

2) Cholinesterase catalyzes the reaction of its substrate acetylcholine to form the intermediate product choline, a substrate for choline oxidase (1.1.3.17), which catalyzes the conversion of choline to trimethyl ammonium acetaldehyde wherein hydrogen peroxide is also produced.

3) Creatinine kinase catalyzes the reaction of its substrate creatinine in the presence of adenosine diphosphate (ADP) to form the intermediate product adenosine triphosphate (ATP), which in the presence of hexokinase causes the conversion of glucose to the intermediate glucose-6-phosphate (G-6-P), which in turn is a substrate for the enzyme glucose-6-phosphate dehydrogenase (G-6-PDH), wherein NAD is converted to the intermediate NADH during the catalytic reaction of G-6-P with G-6-PDH; hydrogen peroxide and NAD are produced in the reaction of NADH-FMN oxidoreductase (1.6.99.3) with NADH.

4) Phosphatase catalyzes the reaction of its substrate CH₃CH₂OPO₃H₂ to form the intermediate product ethanol, which in turn is a substrate for the enzyme alcohol dehydrogenase (ADH), wherein NAD is converted to the intermediate product NADH during the catalytic reaction of ethanol with ADH to form acetaldehyde; hydrogen peroxide and NAD are produced in the reaction of NADH-FMN oxidoreductase (1.6.99.3) with NADH.

5) In the catalytic reaction of lactate dehydrogenase with its substrate lactate in the presence of NAD the intermediate product NADH is formed; hydrogen peroxide and NAD are produced in the reaction of NADH-FMN oxidoreductase (1.6.99.3) with NADH.

6) Glucose is an enzyme substrate for glucose oxidase (1.1.3.4), the products being gluconic acid and hydrogen peroxide.

7) Cholesterol is an enzyme substrate for cholesterol oxidase, hydrogen peroxide being formed during the reaction.

8) Creatinine is an enzyme substrate for creatinine amidinehydrolase, which produces the intermediate product sarcosine, which in turn is an enzyme substrate for the enzyme sarcosine oxidase (1.5.3.1), the products being formaldehyde and hydrogen peroxide.

9) Uric acid is an enzyme substrate for uricase, hydrogen peroxide being formed during the catalytic reaction.

Hydrogen peroxide is also produced by certain types of cells and, thus, the invention permits the detection of hydrogen peroxide as an indicator of cellular activity. Hydrogen peroxide is also produced by substrates for oxidase enzymes in the catalyzed reaction of the substrate with molecular oxygen. In such a reaction the substrate is oxidized and is the hydrogen donor and molecular oxygen is the acceptor.

In one aspect, the present invention is directed to reagent for use in an assay where a peroxidase is part of a signal producing system. The reagent includes a peroxide, a chromogen, and sodium azide. When the peroxidase substrate solution is added to a reaction mixture including the sample contaminated with interfering peroxidases (those other than peroxidase of the reagent), color produced by the interfering peroxidases is suppressed without substantial reduction in the amount of color by activity of the peroxidase (of the reagent) and the chromophoric substrate.

Sodium azide is employed in the invention in an inhibitory amount, i.e., in an effective amount to inhibit the action of endogenous enzymes in the sample but not to the extent that it will eliminate the activity of the peroxidase that is part of the signal producing system. The azide may be added directly to the sample, or added first to a reagent employed in the assay. The azide may be added before the assay begins, or after the addition of one or more reagents, but of course is preferably added before the endogenous enzymes have the opportunity to act on the peroxide present in the assay system.

In another aspect, the concentration of azide in the substrate solution as low as about 3 μM can produce substantial suppression of interference and as much as about 1000 μM can be used with substantial retention of HRP signal. In one aspect of the invention, the concentration of azide in the reagent is about 3 μM to about 500 μM. In another aspect, the concentration is about 50 μM to about 500 μM. In general, while higher concentrations of azide can be expected to suppress the signal of all peroxidases present in the assay, the azide will inhibit the signal of the plant peroxidases to a lesser extent than the interfering peroxidases. Therefore, even when the signal of the peroxidase that is part of the signal producing system is inhibited, because the background signals produced as a result of the interfering peroxidases have been substantially eliminated, a valid result from an assay using the method can be obtained.

In another aspect, the pH of the substrate solution is buffered to a pH of 3 - 10. A pH of between 5.0 and 6.0 minimizes the volatility of azide, which is converted by acid to hydrazoic acid with an equilibrium constant, expressed as pK, of 4.72. At pH 4.72 azide will be expected to be 50% converted to hydrazoic acid that has a boiling point of 37° C. Therefore, at 37° C. (and to a lesser extent below this temperature) azide will be converted to a vapor that can escape from an open container. Above pH 5.0 only a small fraction of azide is expected to be in a vapor form and most of the azide will remain in solution.

Above pH 6 the activity of some peroxidase substrates declines. Because azide is reported to be less effective as an inhibitor of HRP above pH 6, an increase in pH may improve the selectivity of azide. One-half to two-thirds of azide evaporates in less than a half hour from a microwell containing 100 μL of 5 mM azide in succinate buffer below pH 4.5, but at pH 5.0 only one-third of the azide is lost. At pH 5.5 and above there is no significant loss of azide over a half hour. It is reported that no azide is lost from closed containers, even down to pH 3, so the reagent of the invention is stable if kept enclosed or similarly covered during analysis over an unlimited time of analysis. Incubation times longer than 30 minutes have not been very useful in peroxidase-based analysis due to substrate evaporation and gradual inactivation of peroxidase by peroxide, which occurs naturally after a limited number of catalytic cycles.

In one aspect of the invention, the peroxidase substrate solution contains per liter, 3 mmol of beta-hydroxypropylcyclodextrin, 2 mmol of TMB (3,3′,5,5′-tetramethylbenzidine), 2 mmol of hydrogen peroxide, 20 mL of DMF (N,N-dimethylformamide), and 50 mmol of sodium succinate, pH 5.6, and 100 μmol of sodium azide. Reaction of the substrate with a sample containing HRP produces substantial blue color that is proportional to the amount of HRP in the sample, and not at all in proportion to the amount of interfering peroxidases in the sample.

Organic solvent does not effect the function of azide, and 0.1-20% of commonly used solvents such as dimethylformamide, methylpyrrolidinone, dimethylsulfoxide, hydroxypropylcyclodextrins, alkylcelluloses, glycols, glycol ethers, polyvinyl alcohol, polyvinylpyrrolidone, surfactants such as alkylpolyoxyethylene ethers, alkylsufates, alkylsulfonates, alkylfluorocarbons, alkylsiloxanes and the like may be used. Solvent modifiers such as salts in the amount of 1-1000 mmol per liter such as alkali earth and alkali metal halide, sulfate, and phosphate salts are tolerable.

Azide is chelated by transition metal ions, such as ferric, cupric, chromic and manganous ions, but these are strong oxidants that degrade peroxidase chromogens and are usually excluded from peroxidase substrates. Non-oxidizing transition metal ions such as zinc, nickel, cobalt and the like, may chelate azide, reducing its effectiveness. These have not been reported as constituents of peroxidase substrates but rather may be present as contaminants.

The buffer used for pH adjustment does not restrict the effect of the invention when used in amounts between 10-100 mM. Buffers such as acetate, citrate, succinate or other carboxylic acids are suitable, as are phosphate, amino acids such as gamma-aminobutyric acid, aminoalkyl sulfonates such as N-morpholinoethanesulfonate, imidazole, or ethylenediamines.

Antioxidants are often included in peroxidase substrates, such as EDTA, EGTA, DTPA, biquinolines, bipyridines, pyridyltriazines and the like, and these do not affect the invention. These chelators may be usefully employed to release azide from transition metal complexes.

In another aspect, the invention is directed to method of reducing interference of at least one of eosinophil peroxidase, myeloperoxidase and hemoglobin in an assay of a biological sample having horseradish peroxidase as a component of a signal producing system. The method includes adding to the sample a reagent comprising hydrogen peroxide, a chromogen and sodium azide. HRP may be added to the sample as part of a conjugate that includes a binding partner for the analyte (e.g. in a sandwich type immunoassay) or an analyte analog (e.g. in a competitive immunoassay). Analyte analogs are substances that are similar to the analyte but are suitable for conjugation to HRP. In some instances the analyte analog is the analyte and may be bound directly to HRP.

Various techniques for conjugating peroxidases to other elements a signal producing system are well known to those skilled in the art. For the purposes herein, conjugating is synonymous with attaching, so that a peroxidase may be conjugated, or attached to, a binding partner (or other element of a signal producing system) through an intermediate, such a particle. The conjugating includes the immobilization-of both a peroxidase and binding partner to a particle or other substance. In addition, the peroxidase and the binding partner may themselves be conjugated to separate members of a separate specific binding pair, such as biotin and streptavidin, and the attachment of the peroxidase to the binding partner is through the binding of the members of the separate specific binding pair.

In another aspect, the invention includes a method of detecting an analyte in a sample. The method includes conjugating HRP to a specific binding partner for the analyte and contacting the conjugate with the sample to form a complex comprising the conjugate bound to the analyte. The complex is separated from unbound conjugate. Many ways are known in the art to accomplish the separation, most notably through the attachment of the complex to a solid phase, usually as the result of presence or absence of an analyte in the sample. Once the complex is attached to the solid phase, the unbound conjugate can be removed by washing the solid phase or other known methods. The complex is then contacted with reagent comprising a peroxide, a chromogen and sodium azide. A signal produced as a result of the activity of the HRP can be detected, thereby allowing the detection of the analyte in the sample. In another aspect, the HRP is attached to an analyte analog and contacted, along with the sample, to a binding partner to the analyte. The amount of a complex of the conjugate and the binding partner for the analyte that is formed will depend upon the amount of analyte in the sample. Unbound conjugate is separated from the complex. A signal produced as a result of the activity of the HRP determines the presence, absence or amount of analyte in the sample.

The invention is not intended to be limited to any specific analyte detection method, so long as the method uses a peroxidase as a component of a signal producing system. Detection methods, such as immunoassays and histological methods, and various formats of solution based and solid phase chemistries, are well known to those of skill in the art. Also well known are methods of designing and making reagents and devices including specific binding partners for the analytes or other components of the system. The invention is not intended to be limited to any device for use in conducting the detection method. Any device that can incorporate a peroxidase as a member of a signal producing system, for example microplates and lateral flow devices, are within the scope of the invention. In one aspect, the invention uses the device described in U.S. Pat. Nos. 5,726,010 and 6,436,722 which are incorporated by reference herein in their entirety.

The following are provided for exemplification purposes only and are not intended to limit the scope of the invention described in broad terms above. All references cited in this disclosure are incorporated herein by reference.

### EXAMPLES

### Example 1

### Suppression of Interfering Peroxidase Activity Improves Accuracy

SLF5 glass fiber discs, 6.4 mm diameter (Whatman, Inc.) were sensitized with 13 μL of 1 mg/mL rabbit anti-heartworm antibody containing 0.7% sodium bicarbonate, 2% sucrose, and 30 μg/mL FDC Blue 1 dye. Heartworm antibody can be produced from heartworm antigen prepared by disrupting a parasitic worm in an aqueous buffer solution, centrifuging to remove insoluble and particulate components, adding of trichloroacetic acid to a concentration of 15% w/v, centrifuging to remove insoluble components, dialyzing against an aqueous buffer solution and lyophilizing. This antigen preparation is used to produce antibodies in mammals using standard methods (see, e.g., Harlow, E. and D. Lane, 1988, “Antibodies: A Laboratory manual”, Cold Spring Harbor Laboratory, Cod Spring Harbor, N.Y., USA, ISBN 0-87969-314-2 pp 92-286).

The discs were washed with buffer containing, per liter, 25 mmol of 3-(N-morpholino)-2-hydroxypropane sulfonic acid MOPSO, 100 mmol of NaCl, 1 g of Brij® 35 and 1 g of tetrasodium EDTA, pH 7.4. Two 15 μL drops of a mixture of 100 μL of whole canine blood containing 950 eosinophils per μL (normal range: 100-1500 eosinophils per μL), 0.5%-25% v/v of heartworm extract in fetal bovine serum and 25 μL of 100 μg/mL of chicken anti-heartworm HRP-conjugated antibody was added to the glass fiber disc and allowed to incubate for 5 minutes at 23C. The discs were washed with a buffered bland protein solution containing 1% Triton X-100 and transferred to polystyrene microwells.

A peroxidase substrate containing 100 μmol per liter of sodium azide, was added to the discs and allowed to incubate for 10 minutes, after which time the amount of blue color produced was measured by bichromatic absorbance at 650 nm-562 nm, compensating the background contribution from the glass fiber matrix. Duplicate discs were also processed in an identical manner except these were incubated instead with the peroxidase substrate but lacking in azide. Table 1 shows that when the peroxidase substrate lacking azide is used, the interference by endogenous canine peroxidase completely obscures the dose-response, producing a poor correlation (r<0.2) of absorbance response at 650 nm with the amount of added heartworm antigen. On the other hand, when azide is added to the same substrate, a dose-response is clearly discernible and the amount of color is correlated with the amount of antigen (r>0.96).

TABLE 1

| Amount of | No azide | azide added |
|---|---|---|
| Antigen | mA650-562 | mA650-562 |
| 25% | 538 | 305 |
| 10% | 597 | 179 |
| 5% | 504 | 149 |
| 2.5% | 571 | 147 |
| 1% | 428 | 123 |
| 0.5% | 543 | 97 |
| 0% | 516 | 118 |
| buffer blank | 78 | 64 |

### Example 2

### Determination of Inhibition of HRP by Azide

To determine the interference as a result of the azide alone, the immunoassay of Example 1 was conducted, except that the analyte, heartworm antigen, is added to serum (instead of whole blood) which does not contain interfering peroxidases. Table 2 shows the results of assays using a TMBlue® (Intergen Company, Purchase, N.Y.), a soluble peroxidase substrate. Table 3 shows the same assay using TMBM (Moss Inc, Pasadena, Md.), a precipitating peroxidase substrate. The TMBlue® substrate was incubated with the glass fiber discs for 3 minutes and TMBM substrate was incubated for 15 minutes until bichromatic measurement at 650 mn-562 nm. With both substrates, azide does not produce a significant diminution of assay sensitivity up to 100 μM, and even at 500 μM azide the decrease in sensitivity is not severe and allows detection of even the lowest antigen calibrator by both substrates (5% value is 2-fold higher than blank value.

TABLE 2
TMBlue ® Substrate

| | no | 50 μM | no | 100 μM | no | 500 μM |
|---|---|---|---|---|---|---|
| Heartworm | azide | azide | Azide | azide | Azide | Azide |
| Antigen | (mA) | (mA) | (mA) | (mA) | (mA) | (mA) |
| 100% | 707 | 646 | 911 | 1046 | 1014 | 734 |
| 50% | 399 | 408 | 570 | 628 | 627 | 462 |
| 25% | 263 | 270 | 370 | 395 | 346 | 265 |
| 10% | 107 | 119 | 184 | 150 | 166 | 121 |
| 5% | 70 | 64 | 104 | 110 | 102 | 73 |
| 0% | 22 | 22 | 42 | 31 | 30 | 21 |

TABLE 3
TMBM Substrate

| | no | 50 μM | no | 100 μM | no | 500 μM |
|---|---|---|---|---|---|---|
| Heartworm | azide | azide | Azide | azide | Azide | Azide |
| Antigen | (mA) | (mA) | (mA) | (mA) | (mA) | (mA) |
| 100% | 784 | 776 | 803 | 800 | 822 | 491 |
| 50% | 502 | 535 | 544 | 511 | 495 | 363 |
| 25% | 346 | 264 | 362 | 324 | 314 | 173 |
| 10% | 198 | 196 | 220 | 197 | 186 | 112 |
| 5% | 126 | 138 | 163 | 123 | 111 | 70 |
| 0% | 44 | 46 | 69 | 73 | 52 | 37 |

### Example 3

### Selectivity of Azide for EPO, MPO, and Hgb over HRP

Assays were run to determine the selectivity of azide for interference from EPO, MPO and hemoglobin. Activities were measured in solution using a peroxidase substrate consisting of, per liter, 1.4 mmol of TMB, 0.3 mmol of hydrogen peroxide, 20 mmol of sodium chloride, 30 mg of Triton X-100, and 100 g of sucrose. HRP was a conjugate of chicken antibody (used in Example 1). MPO and EPO were essentially homogeneous commercial preparations isolated from human leukocytes. Hemoglobin (Hgb) was obtained as a Triton X-100 lysate of feline erythrocytes. The results are reported in Table 4.

TABLE 4
% of control activity (without azide)

| | | HRP | MPO | EPO | Hgb |
|---|---|---|---|---|---|
| 3 | μM azide | 101 | 63.1 | 13.4 | 99.7 |
| 30 | μM azide | 83.5 | 15.5 | 0.7 | 26.4 |

Although various specific embodiments of the present invention have been described herein, it is to be understood that the invention is not limited to those precise embodiments and that various changes or modifications can be affected therein by one skilled in the art without departing from the scope and spirit of the invention.

1. A reagent for use in a detection method having peroxidase as a component of a signal producing system comprising hydrogen peroxide in a concentration of about 2 mM, a chromogen and sodium azide in a concentration from about 20 μM to about 1000 μM.

2. (Cancelled)

3. (Cancelled)

4. (Cancelled)

5. (Cancelled)

6. (Cancelled)

7. (Cancelled)

8. The reagent of claim 1 wherein the sodium azide is in a concentration from about 20 μM to about 500 μM.

9. The reagent of claim 1 wherein the sodium azide is in a concentration from about 50 μM to about 500 μM.

10. A signal producing system for use in a method for detecting an analyte in a sample, the system comprising a first peroxidase, hydrogen peroxide in an amount of about 2 mM, a chromogen and sodium azide in a concentration from about 3 μM to about 500 μM, wherein the sodium azide substantially inhibits the activity of endogenous peroxidases in the sample but does not substantially inhibit the activity of the first peroxidase.

11. (Cancelled)

12. The signal producing system of claim 10 wherein the first peroxidase is horseradish peroxidase.

13. (Cancelled)

14. A method of reducing interfering peroxidase activity in an assay for detecting an analyte, wherein the assay uses a peroxidase as a component of a signal producing system, the method comprising adding to the assay a reagent comprising a substrate for the peroxidase and sodium azide in a concentration from about 3 μM to about 1000 μM.

15. (Cancelled)

16. The method of claim 14 wherein the azide is in a concentration from about 3 μM to about 500 μM.

17. The method of claim 14 wherein the reagent further comprises hydrogen peroxide.

18. The method of claim 17 wherein the hydrogen peroxide is in a concentration of about 2 mM.

19. A method of reducing interference of at least one animal peroxidase in an assay of a biological sample having horseradish peroxidase as a component of a signal producing system, the method comprising adding to the assay a reagent comprising hydrogen peroxide, a chromogen and sodium azide in a concentration from about 3 μM to about 1000 μM.

20. The method of claim 19 wherein the at least one animal peroxidase is at least one of eosinophil peroxidase, myeloperoxidase, and hemoglobin.

21. A method of detecting an analyte in a sample comprising:
conjugating horseradish peroxidase (HRP) to a first specific binding partner for the analyte;
providing a solid phase having bound thereto a second specific binding partner for the analyte, wherein the first and the second specific binding partners for the analyte may be the same or different;
contacting the conjugate with the sample and the solid phase to form a complex comprising the analyte and the conjugate bound to the solid phase;
separating the complex from unbound conjugate;
contacting the complex with a reagent comprising a peroxide, a chromogen and sodium azide; and
detecting a signal produced as a result of the activity of the HRP in the presence of the sodium azide, thereby detecting the analyte in the sample.

22. A method of detecting an analyte in a sample comprising:
conjugating horseradish peroxidase (HRP) to an analog of the analyte;
contacting the conjugate with the sample and a specific binding partner for the analyte to form a complex of the conjugate bound to the binding partner;
separating the complex from unbound conjugate;
contacting the complex with a reagent comprising a peroxide, a chromogen and sodium azide; and
detecting the presence, absence or amount of a signal produced as a result of the activity of the HRP in the presence of the sodium azide, thereby detecting the analyte in the sample.

23. A kit for detecting an analyte in a sample comprising (a) an immunoassay device comprising a solid phase and (b) a reagent comprising a peroxide, a chromogen and sodium azide in a concentration from about 3 μM to about 1000 μM.

24. The kit of claim 23 further comprising a first specific binding reagent that specifically binds the analyte or an analog of the analyte.

25. The kit of claim 24 wherein the first specific binding reagent is conjugated to the peroxidase.

26. The kit of claim 24 wherein the first specific binding reagent is attached to the solid phase.

27. The kit of claim 26 further comprising a second specific binding reagent that binds the analyte.

28. The kit of claim 27 wherein the second specific binding reagent is conjugated to the peroxidase.

29. The kit of claim 26 wherein the peroxide is hydrogen peroxide in a concentration of about 2 mM.

30. The method of claim 14 wherein the wherein the sodium azide substantially inhibits the activity of interfering peroxidase but does not substantially inhibit the activity of the peroxidase that is a component of the signal producing system.

31. The method of claim 19 wherein the wherein the sodium azide substantially inhibits the activity of the animal peroxidase but does not substantially inhibit the activity of the horseradish peroxidase.

32. The method of claim 19 wherein the hydrogen peroxide is in a concentration of about 2mM.

33. The method of claim 19 wherein the sodium azide is in a concentration from about 3 μM to about 500 μM.

34. The reagent of claim 19 wherein the sodium azide is in a concentration from about 50 μM to about 500 μM.

35. The method of claim 21 wherein the sodium azide is in a concentration from about 3 μM to about 1000 μM.

36. The method of claim 21 wherein the sodium azide is in a concentration from about 3 μM to about 500 μM.

37. The method of claim 21 wherein the sodium azide is in a concentration from about 50 μM to about 500 μM.

38. The method of claim 22 whereing the sodium azide is in a concentration from about 3 μM to about 1000 μM.

39. The reagent of claim 22 wherein the sodium azide is in a concentration from about 3 μM to about 500 μM.

40. The reagent of claim 22 wherein the sodium azide is in a concentration from about 50 μM to about 500 μM.
